(19) United States
(12) Patent Application Publication
(10) Pub. No.: US 2017/0035288 A1
(43) Pub. Date: Feb. 9, 2017

(54) LARGE PATIENT HEAD AND CHIN REST

(71) Applicant: RICK A. NORWOOD, BLUE BELL, PA (US)

(72) Inventor: RICK A. NORWOOD, BLUE BELL, PA (US)

(21) Appl. No.: 15/227,620

(22) Filed: Aug. 3, 2016

Related U.S. Application Data
(63) Continuation-in-part of application No. 14/185,563, filed on Feb. 20, 2014, now Pat. No. 9,128,976.

(51) Int. Cl. A61B 3/00

(52) U.S. Cl. CPC A61B 3/0083; A61B 3/0075

(57) ABSTRACT

An improved eye examination device for use of an eye examination is described herein. The device includes a support device enclosing a chin rest for an eye patient to place their chin in order for an eye professional/technician to examine at least one eye of the eye patient.

### CROSS-REFERENCE

The present application claims benefit of the Provisional Application Ser. No. 62/136,338, entitled “Large Patient Head and Chin Rest” filed on Mar. 20, 2015, and, Nonprovisional application Ser. No. 14/185,563 entitled “Large Patient Head and Chin Rest” filed on Oct. 21, 2015, which both are herein incorporated by reference in their entirety.

### BRIEF DESCRIPTION OF THE FIGURES

For a more complete understanding of the present invention, including its features and advantages, reference is now made to the detailed description of the invention taken in conjunction with the accompanying drawing in which:

FIG. 1 illustrates a front view of an embodiment of present invention;

FIG. 2 illustrates a side view of this embodiment of the present invention;

FIG. 3 is rear/side view of this embodiment of the present invention;

FIG. 4 is a detailed view of a portion of the present invention;

FIG. 5 illustrates a front view of another embodiment of present invention;

FIG. 6 illustrates a side view of this embodiment of the present invention;

FIG. 7 is top of this embodiment of the present invention;

FIG. 8 is a top/front view of this embodiment of the present invention;

FIG. 9 is a top/front view of another embodiment of the present invention;

FIG. 10 illustrates a front view of another embodiment of present invention;

FIG. 11 illustrates a rear view of this embodiment of present invention;

FIGS. 12-13 illustrate other front views of this embodiment of present invention;

FIGS. 14-15 illustrate detailed view of the embodiment of the present invention;

FIG. 16 illustrates a front view of another embodiment of the present invention;

FIG. 17 illustrates another view of the embodiment of FIG. 16;

FIG. 18 illustrates a side view of the embodiment of FIG. 16;

FIG. 19 illustrates a top view of the embodiment of FIG. 16; and

FIG. 20 illustrates more details of the embodiment in FIG. 19.

### DETAILED DESCRIPTION OF THE INVENTION

While the making and using of various embodiments of the present invention are discussed in detail below, it should be appreciated that the present invention provides many applicable inventive concepts that may be embodied in a wide variety of specific contexts. The specific embodiments discussed herein are merely illustrative of specific ways to make and use the invention and do not delimit the scope of the invention.

An eye examination is a series of tests performed by an ophthalmologist (medical doctor), optometrist, or orthoptist assessing vision and ability to focus on and discern objects, as well as other tests and examinations pertaining to the eyes. Health care professionals often recommend that all people should have periodic and thorough eye examinations as part of routine primary care, especially since many eye diseases are asymptomatic.

Eye examinations may detect potentially treatable blinding eye diseases, ocular manifestations of systemic disease, or signs of tumors or other anomalies of the brain.

Ideally, the eye examination consists of an external examination, followed by specific tests for visual acuity, pupil function, extraocular muscle motility, visual fields, intraocular pressure and ophthalmoscopy through a dilated pupil.

A minimal eye examination consists of tests for visual acuity, pupil function, and extraocular muscle motility, as well as direct ophthalmoscopy through an undiluted pupil.

Close inspection of the anterior eye structures and ocular adnexa are often done with a slit lamp which is a table mounted microscope with a special adjustable illumination source attached. A small beam of light that can be varied in width, height, incident angle, orientation and color, is passed over the eye. Often, this light beam is narrowed into a vertical “slit”, during slit-lamp examination. The examiner views the illuminated ocular structures, through an optical system that magnifies the image of the eye and the patient is seated while being examined, and the head stabilized by an adjustable chin rest.

This allows inspection of all the ocular media, from cornea to vitreous, plus magnified view of eyelids, and other external ocular related structures. Fluorescein staining before slit lamp examination may reveal corneal abrasions or herpes simplex infection.

The binocular slit-lamp examination provides stereoscopic magnified view of the eye structures in striking detail, enabling exact anatomical diagnoses to be made for a variety of eye conditions.

Also ophthalmoscopy and gonioscopy examinations can also be performed through the slit lamp when combined with special lenses. These lenses include the Goldmann 3-mirror lens, gonioscopy single-mirror/Zeiss 4-mirror lens for (ocular) anterior chamber angle structures and +90D lens, +78D lens, +66D lens & Hruby (−56D) lens, the examination of retinal structures is accomplished.

Intraocular pressure (IOP) can be measured by Tonometry devices. The eye can be thought of as an enclosed compartment through which there is a constant circulation of fluid that maintains its shape and internal pressure. Tonometry is a method of measuring this pressure using various instruments. The normal range is 10-21 mmHg.

Some of these types of eye examinations include placing the chin of a patient on a chin rest within a support device and then measuring the eye sight. However, most chin rests within standard support devices do not adequately support larger patients. Thus, this type of patient feels uncomfortable when the chin is placed on the chin rest of the device. For example, bigger patients have to squeeze their upper body into the support device in order to place their chin on the chin rest which can be very uncomfortable to the patient.

Therefore, a need exists for a chin rest within a support device that is comfortable for larger patients, as well medium sized patients, to place their chin on the chin rest for the eye examination. The present invention has an advantage to solve this need.

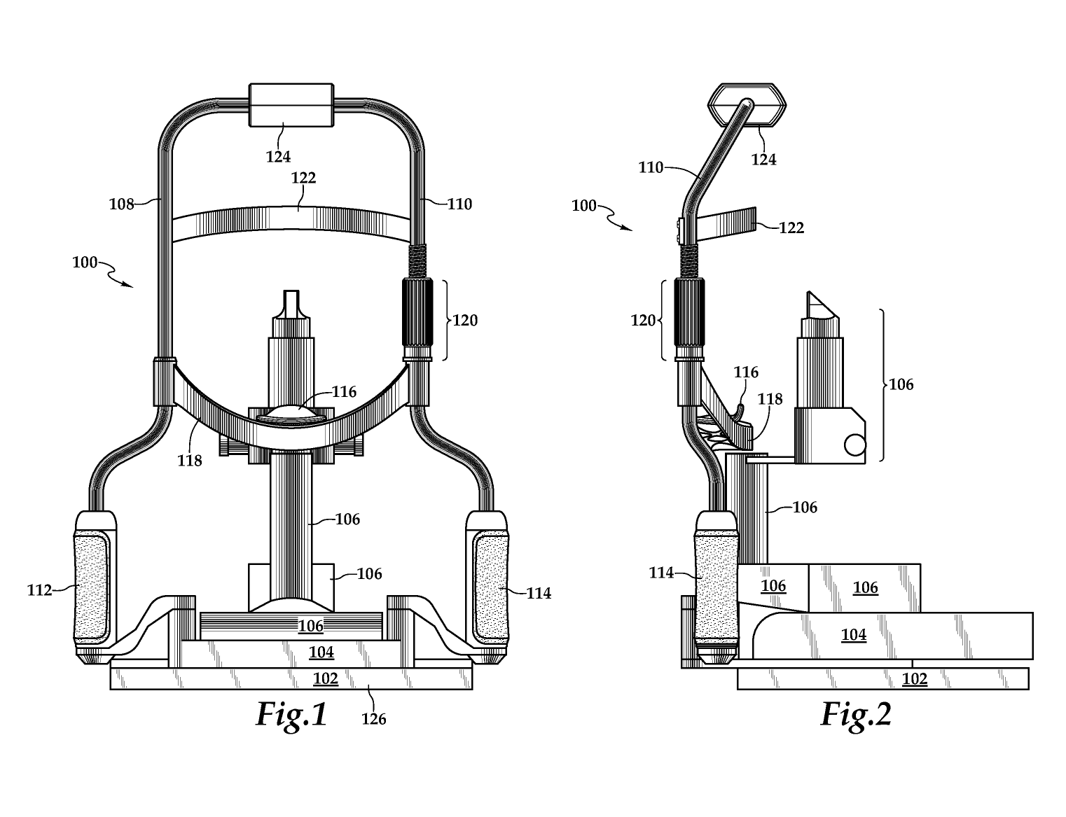

Now turning to FIG. 1, an embodiment of the present invention is illustrated. In this embodiment, a chin rest and head support system 100 is shown attached to table 102. In turn, a support level 104 is attached to the table 102 along with a medical eye testing device 106. The eye testing device 106 is positioned adjacent to the support system 100 in order to allow an eye professional/technician to exam a patient. The support system 100 includes a left support bar 108 as well as a right support bar 110. Also shown in this figure, is a left handle 112 near the bottom of the left support bar 108. Similarly, a right handle 114 near the bottom of the right support bar 110 is shown.

Also shown in this figure is a chin rest 116 attached near the center of curved chin bar 118. The curved chin bar 118 is attached to the right support bar 110 as well as the left support bar 108. At the juncture where the curved chin bar 118 is attached to the right support bar 108, a mechanism 120 allows the curved chin bar 118 to adjust vertically as may be required for the comfort of the patient to be examined. In addition, the head rest 122 is shown attached to the right support bar 110 as well as the left support bar 108. Moreover a handle 124 is shown that may be used to grip the support system 100 in order to reposition or move the support system 100.

FIG. 2 illustrates the support system 100 from a side view. In this figure, the grip 124 is shown attached to the right support bar 110 which includes the adjustment mechanism 120 and the right handle 114. In addition, the head rest 122, the chin rest 116 and curved chin bar 118 along with the eye testing device 106 and the support level 104 are shown.

FIG. 3 illustrates a different perspective of the support system 100 from a rear/side view. In this figure, a chin rest and head support system 100 is shown attached to table 102, which in turn is on top of an additional table 126. However, in other embodiments, the table 102 and the additional table 126 can be combined as well as other configurations of similar supports can be utilized.

Moreover, a support level 104 is attached to the table 102 along with an eye testing device 106. Similar to FIG. 2, the eye testing device 106 is positioned adjacent to the support system 100 in order to allow an eye professional/technician to exam a patient. Additionally, the support system 100 includes the left support bar 108 as well as a right support bar 110. Also shown in this figure, is the left handle 112 near the bottom of the left support bar 108. Likewise, the right handle 114 near the bottom of the right support bar 110 is shown.

Also shown in this figure is the chin rest 116 attached near the center of the curved chin bar 118. The curved chin bar 118 is attached to the right support bar 110 as well as the left support bar 108. At the juncture where the curved chin bar 118 is attached to the right support bar 108, the adjustment mechanism 120 allows the curved chin bar 118 to adjust vertically as shown in FIG. 1. In addition, the head rest 122 is shown attached to the right support bar 110 as well as the left support bar 108. Moreover the handle 124 is shown that may be used to grip the support system 100 in order to reposition or move the support system 100.

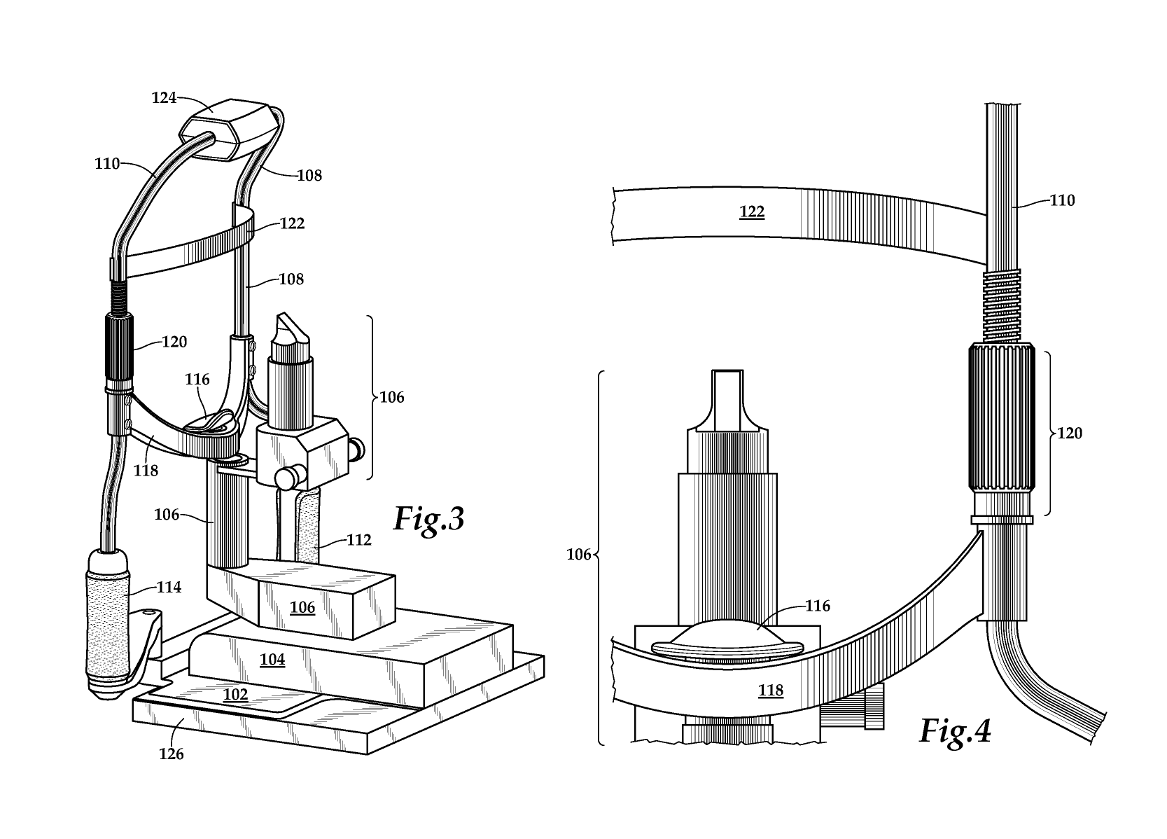

FIG. 4 illustrates a detailed view of the adjustment mechanism 120 that allows the chin rest 116 and the curved chin bar 118 vertically adjust as desired by the eye professional/technician and/or the patient. In this figure, the head rest 122, the right support bar 110, as well as the eye testing device 106 are shown.

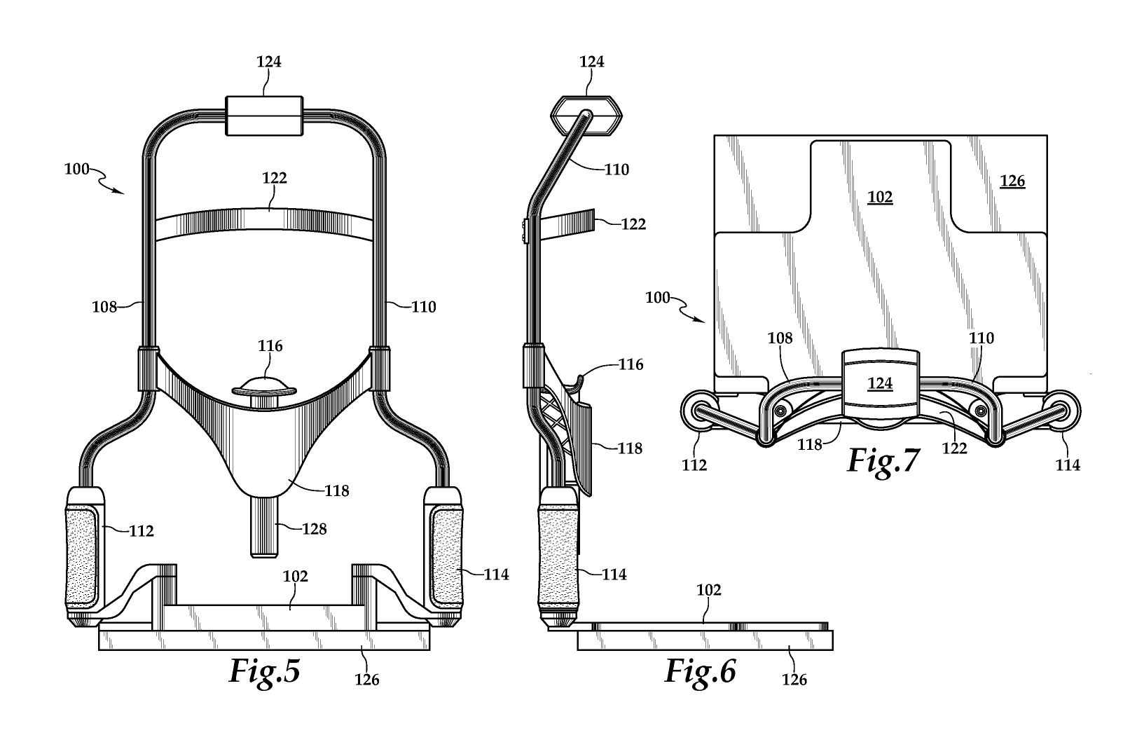

FIG. 5 illustrates another embodiment of the invention. Similar to the first embodiment, the support system 100 includes the left support bar 108, the right support bar 110, the curved chin bar 118, the chin rest 116, the left handle 112, the right handle 114, the head rest 122 and the handle 124. In this embodiment, similarly to FIG. 3, the support system 100 is shown attached to table 102, which in turn is on top of an additional table 126. However, in other embodiments, the table 102 and the additional table 126 can be combined as well as other configurations of similar supports can be utilized. In addition, this embodiment includes an adjustment bar 128 which elevates the chin rest 116 as desired by the eye professional/technician and/or the patient.

FIG. 6 illustrates a side view of embodiment of FIG. 5. Similar to the first embodiment, the support system 100 includes the right support bar 110, the curved chin bar 118, the chin rest 116, the right handle 114, the head rest 122 and the handle 124. In this embodiment, similarly to FIG. 5, the support system 100 is shown attached to table 102, which in turn is on top of an additional table 126. Moreover, although not shown, this embodiment includes the adjustment bar 128 which elevates the chin rest 116 as desired by the eye professional/technician and/or the patient.

FIG. 7 illustrates a top view of embodiment of FIG. 5. This figure illustrates the support system 100 which includes the right support bar 110, the left support bar 112, the curved chin bar 118, the chin rest 116, the right handle 114, the left handle 112 the head rest 122 and the handle 124. Additionally, the support system 100 is shown attached to table 102, which in turn is on top of an additional table 126. Moreover, although not shown, this embodiment includes the adjustment bar 128 which elevates the chin rest 116 as desired by the eye professional/technician and/or the patient.

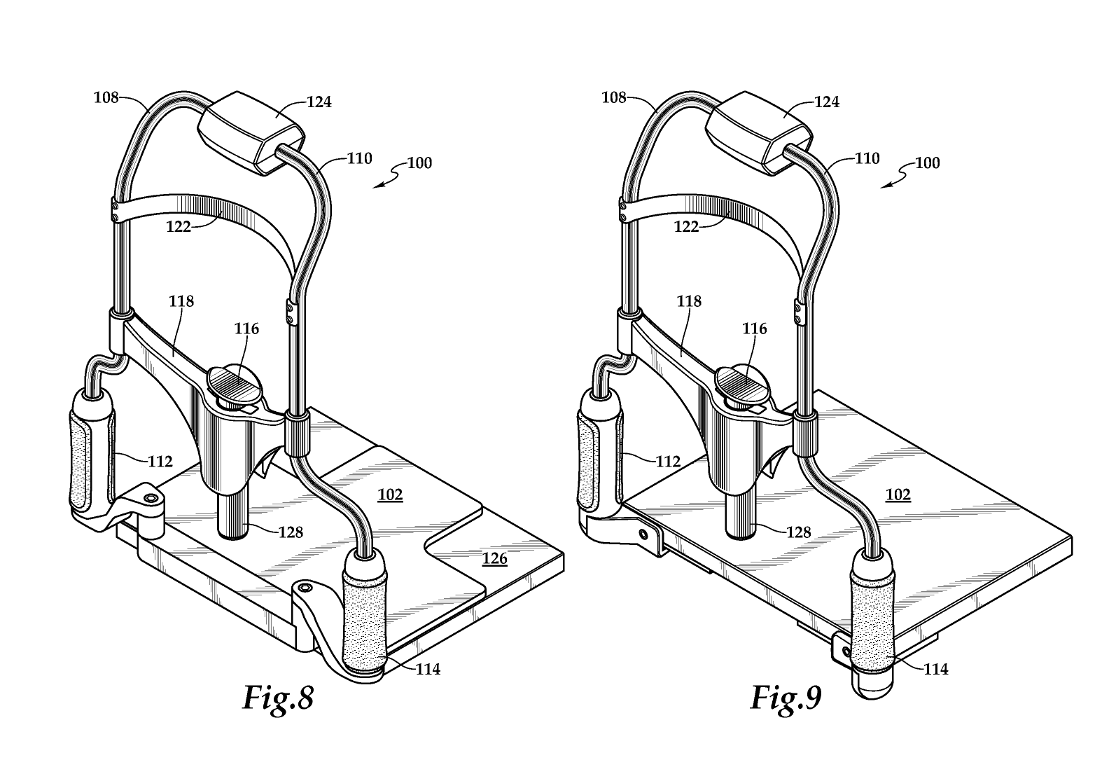

FIG. 8 illustrates another perspective of this embodiment from a top/front view. In this figure, the support system 100 includes the left support bar 108, the right support bar 110, the curved chin bar 118, the chin rest 116, the left handle 112, the right handle 114, the head rest 122 and the handle 124. Like FIG. 5, the support system 100 is shown attached to table 102, which in turn is on top of an additional table 126. However, in other embodiments, the table 102 and the additional table 126 can be combined as well as other configurations of similar supports can be utilized. In addition, this embodiment includes an adjustment bar 128 which elevates the chin rest 116 as desired by the eye professional/technician and/or the patient.

FIG. 9 illustrates a perspective of another embodiment of the present invention from a top/front view. In this figure, the support system 100 includes the left support bar 108, the right support bar 110, the curved chin bar 118, the chin rest 116, the left handle 112, the right handle 114, the head rest 122 and the handle 124. In this embodiment, the support system 100 is shown attached to table 102. However, this embodiment does include the adjustment bar 128 which elevates the chin rest 116 as desired by the eye professional/technician and/or the patient.

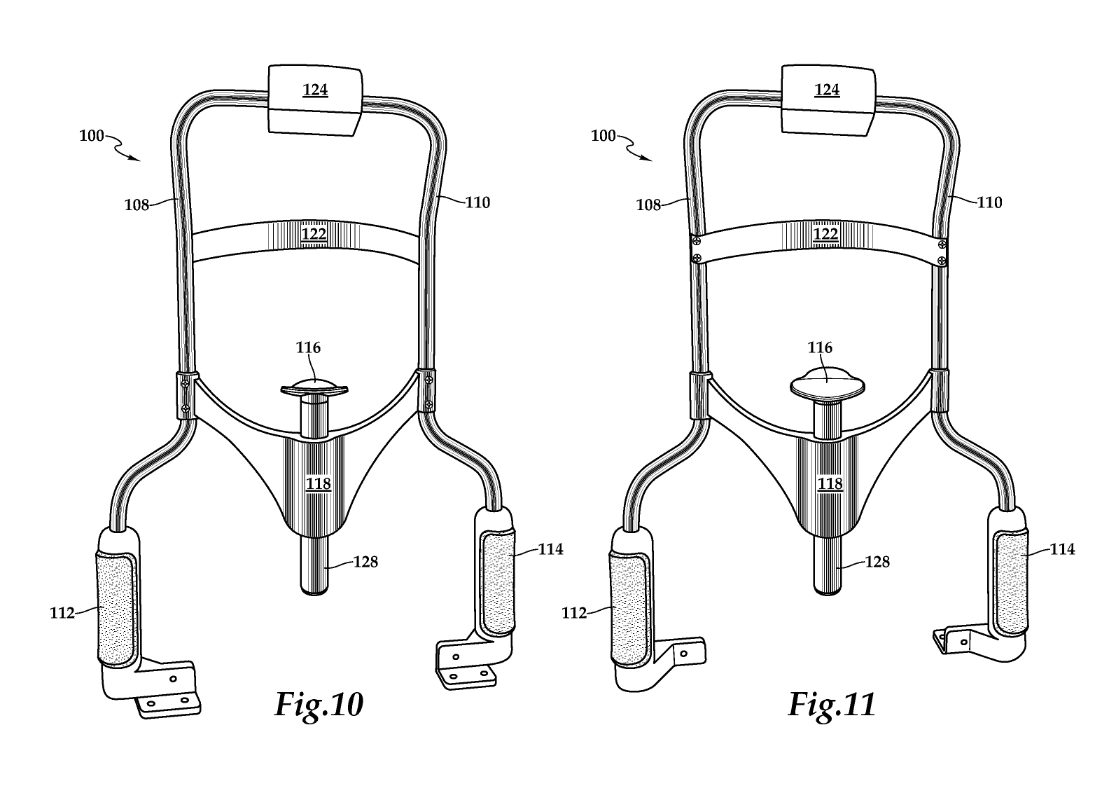

FIG. 10 illustrates a front view of an embodiment of the invention similar to the previously described embodiment. In this embodiment, the support system 100 includes the handle 124, the left support bar 108, the right support bar 110, the curved chin bar 118, the chin rest 116, the left handle 112, the right handle 114, the head rest 122 and the handle 124. Moreover, this embodiment includes an adjustment bar 128 which elevates the chin rest 116 as desired by the eye professional/technician and/or the patient. However, this embodiment shows the bottom of the right 112 and the left 114 handles illustrating attachment configurations that stabilize the support system 100 to a table and eye testing device (both not shown).

FIG. 11 illustrates a rear view of an embodiment of the invention similar to the previously described embodiment. In this embodiment, the support system 100 includes the handle 124, the left support bar 108, the right support bar 110, the curved chin bar 118, the chin rest 116, the left handle 112, the right handle 114, the head rest 122 and the handle 124. Furthermore, this embodiment includes an adjustment bar 128 which elevates the chin rest 116 as desired by the eye professional/technician and/or the patient. However, this embodiment shows the bottom of the right 112 and the left 114 handles illustrating attachment configurations that stabilize the support system 100 to a table and eye testing device (both not shown).

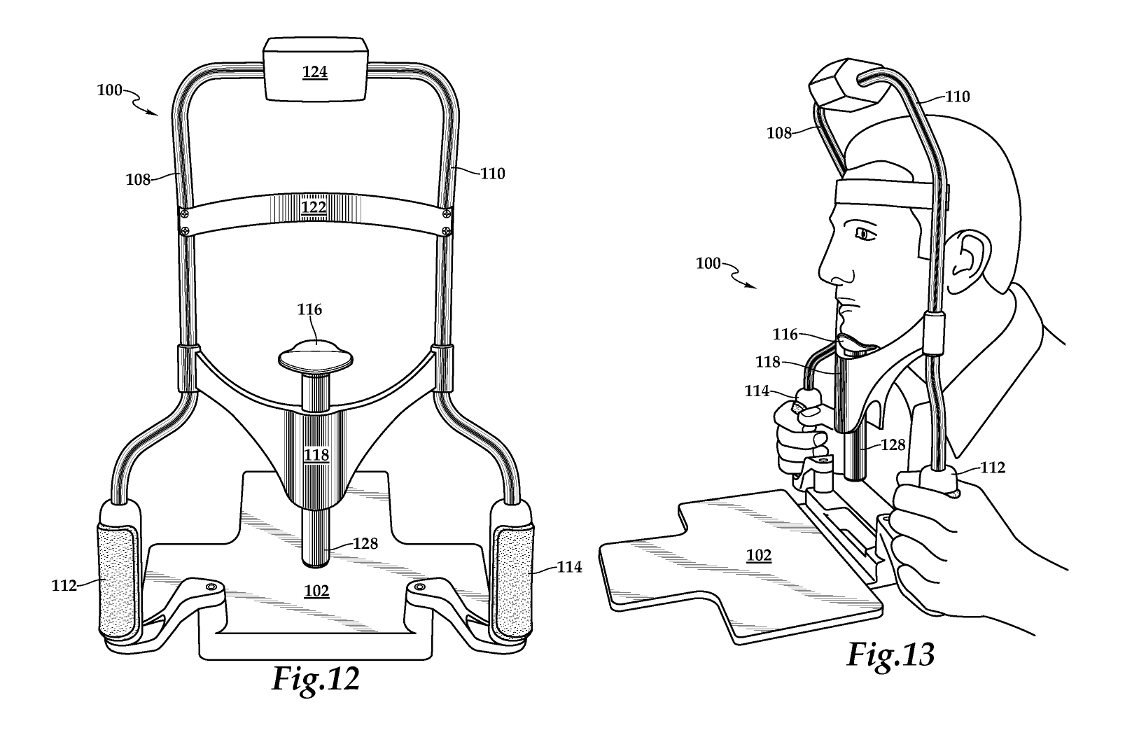

FIG. 12 illustrates a front/top perspective of this embodiment of the invention. In this figure, the support system 100 includes the left support bar 108, the right support bar 110, the curved chin bar 118, the chin rest 116, the left handle 112, the right handle 114, the head rest 122 and the handle 124. However in this figure, the support system 100 is shown attached to table 102. Further, this embodiment includes an adjustment bar 128 which elevates the chin rest 116 as desired by the eye professional/technician and/or the patient.

FIG. 13 shows the support system of FIG. 12 with a patient with his chin positioned in the chin rest as well as his forehead resting on the head rest in preparation to have his eyes examined by an eye professional/technician.

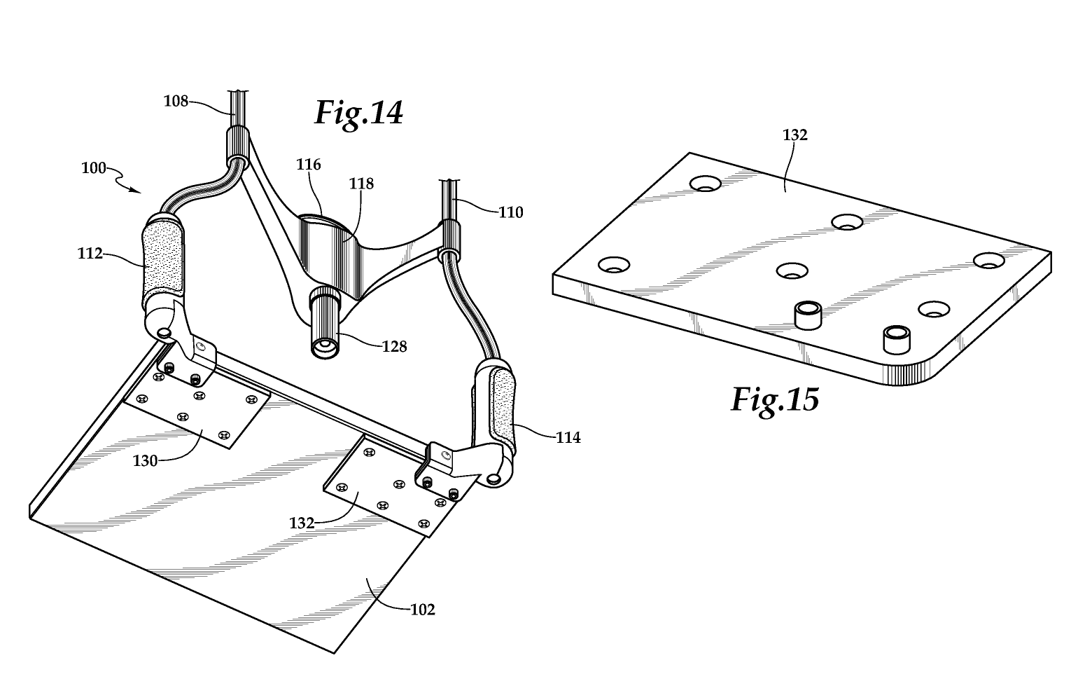

FIG. 14 illustrates a support system 100 with the left support bar 108 and the left handle 112 along with the right support bar 110 and the right handle 114 attached to the table 102. The right 114 and left 112 handles are attached the right 132 and left 130 attachment plates that are used to secure the support system 100 to a table 102.

FIG. 15 illustrates a closer view of the right attachment plate 132 with a sample configuration that can be used to attach the right support bar 110 to the table 102.

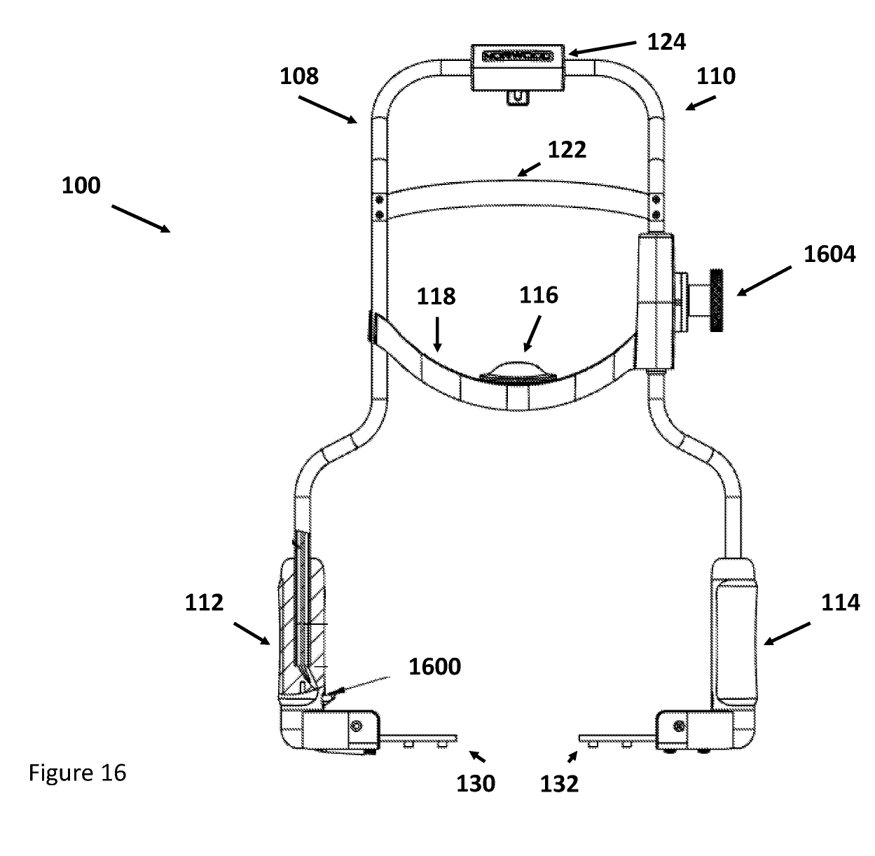

FIG. 16 illustrates another embodiment of the present invention. In this Figure, another embodiment of the present invention is illustrated. In this embodiment, a chin rest and head support system 100 is shown. The support system 100 includes a left support bar 108 as well as a right support bar 110. Also shown in this figure, is a left handle 112 near the bottom of the left support bar 108. Similarly, a right handle 114 near the bottom of the right support bar 110, and a right 132 and a 130 attachment plates that are used to secure the support system 100 to a table 102.

Also shown in this figure is a chin rest 116 attached near the center of curved chin bar 118. The curved chin bar 118 is attached to the right support bar 110 as well as the left support bar 108. At the juncture where the curved chin bar 118 is attached to the right support bar 108, a rotating adjustment mechanism 1604 allows the curved chin bar 118 to adjust vertically as may be required for the comfort of the patient to be examined. In addition, the head rest 122 is shown attached to the right support bar 110 as well as the left support bar 108. Moreover, a handle 124 is shown that may be used to grip the support system 100 in order to reposition or move the support system 100. Further, although not shown in this figure, electrical circuitry is positioned within the handle 124 that can be used to attach an eye examination system, also not shown in this figure. In addition, a cable 1600 attaches to the electrical circuitry and follows within the support bar 108 and extends through the bottom of the support bar 108.

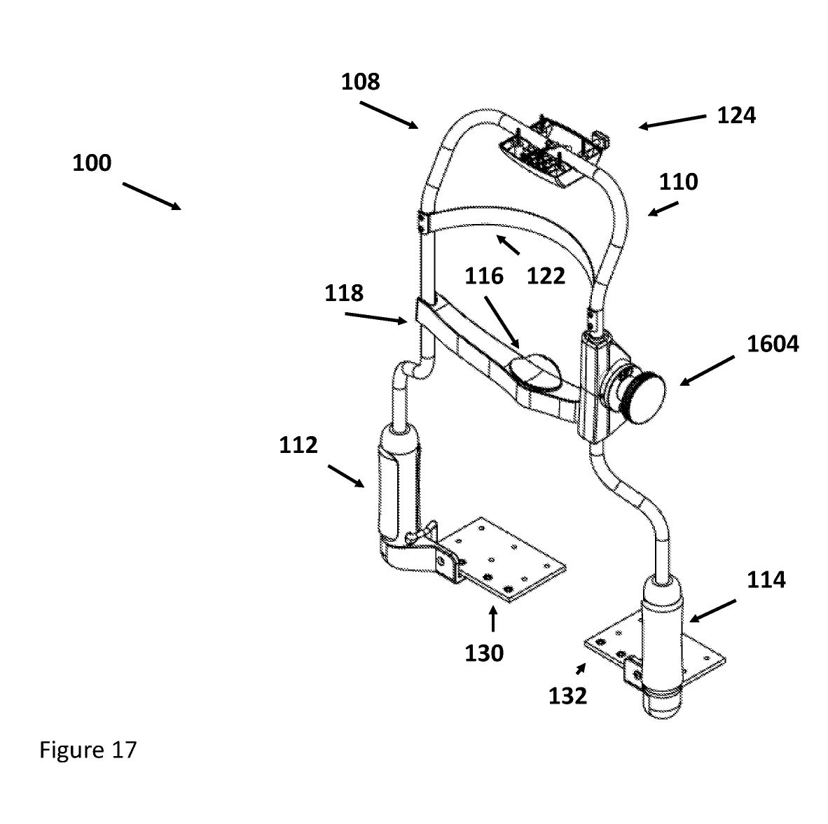

FIG. 17 illustrates another view of the embodiment of FIG. 16. In this Figure, the top of the handle 124 has been removed to show the interior of the handle 124 where the electrical circuitry can be positioned. Similar to FIG. 16, the support system 100 includes the support bars 108, 110, the head rest 122, the curved chin bar 118, the chin rest 116, the rotating adjustment mechanism 1604, the left handle 112, the right handle 114 and the right 132 and left 130 attachment plates that are used to secure the support system 100 to a table (not shown in this figure).

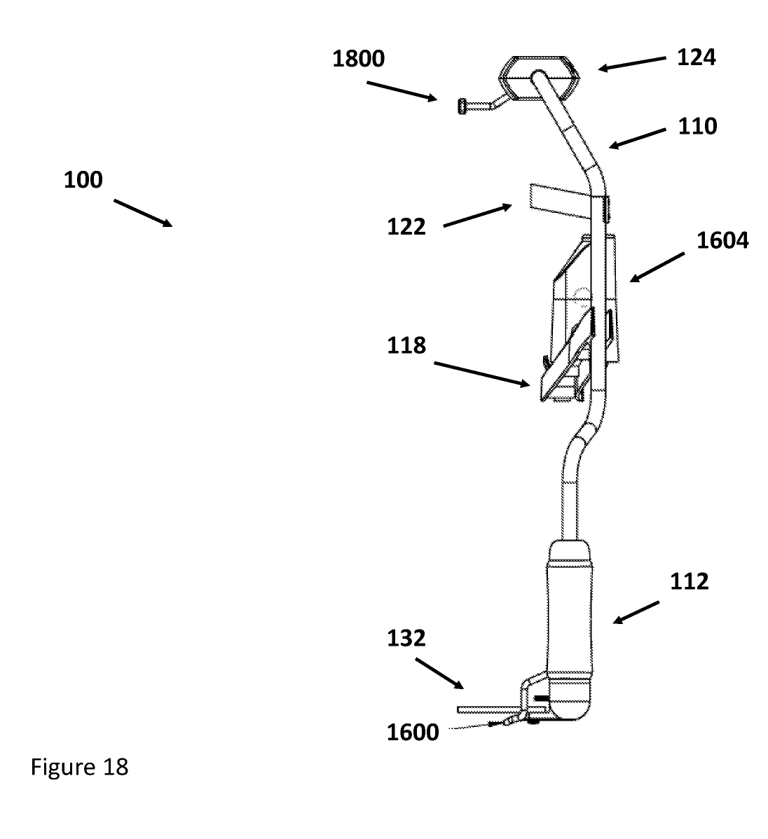

FIG. 18 illustrates a side view of the embodiment of FIG. 16. In this Figure, support system 100 includes the handle 124 with an extension 1800 that connects to an eye examination system (not shown), support bar 108, head rest 122, chin rest 118, rotating adjustment mechanism 1604, left handle 112, attachment plate 132 and the cable 1600.

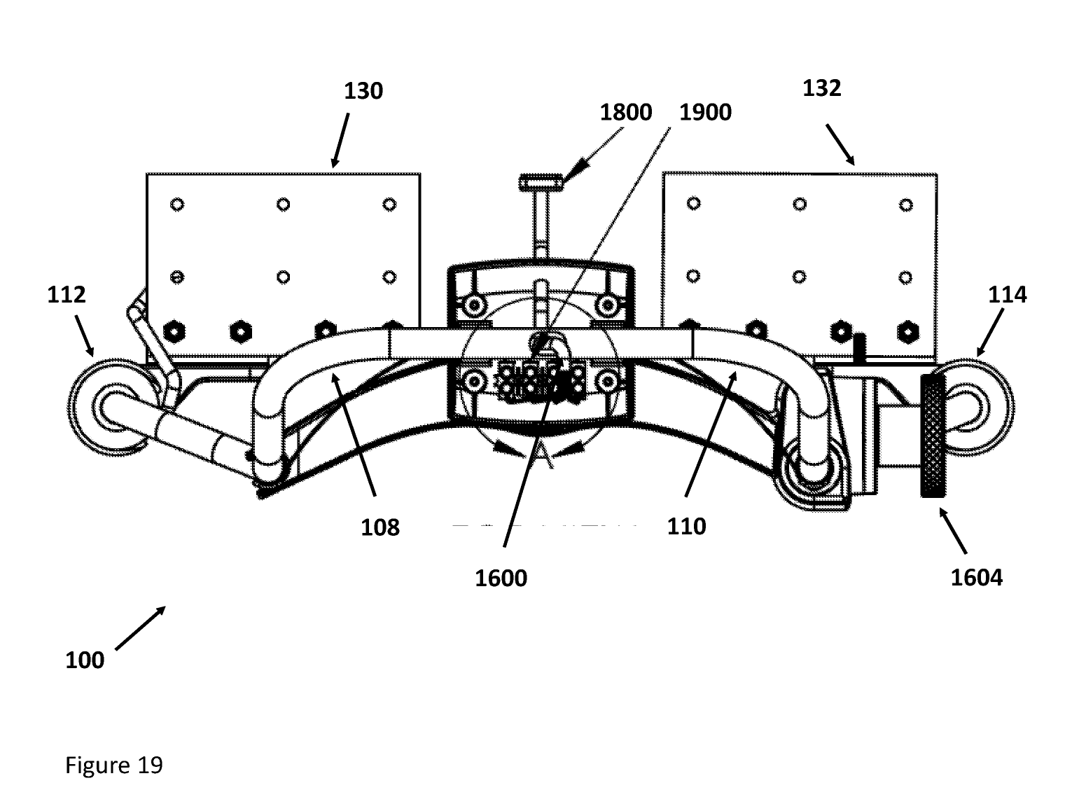

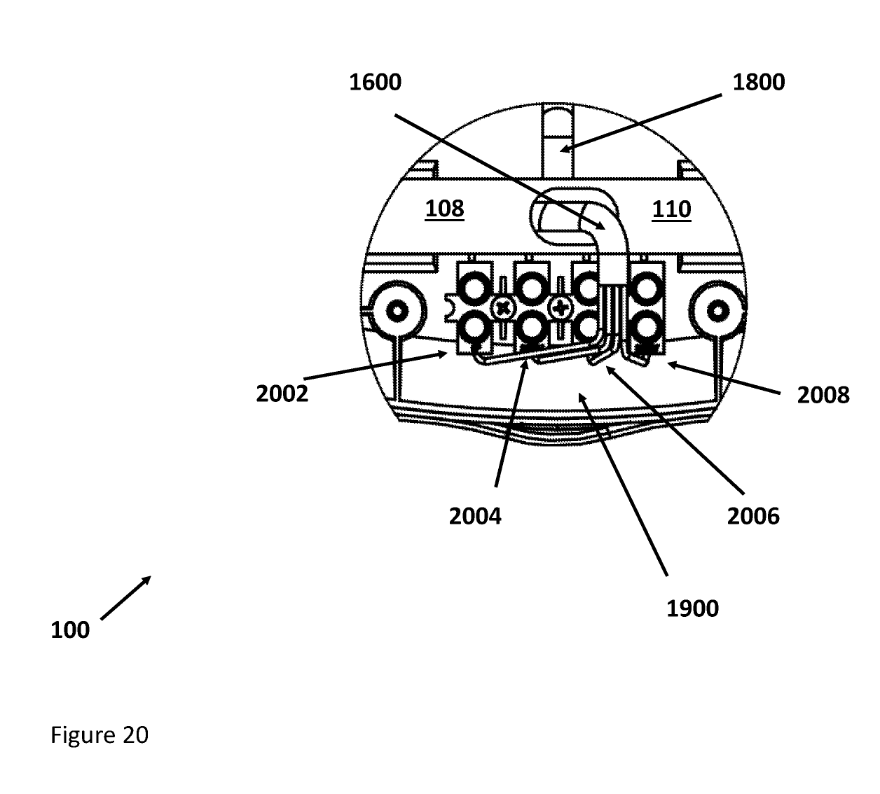

FIG. 19 illustrates a top view of the embodiment of FIG. 16. In this Figure, support system 100 includes the handle 124 with an extension 1800 that connects to an eye examination system (not shown), support bars 108, 110, head rest 122, rotating adjustment mechanism 1604, left handle 112 and right handle 114, attachment plates 130. 132 and the cable 1600 connecting to the extension 1800. In addition, the top of handle 124 is removed to illustrate the electrical circuitry 1900, shown as cross-section A in FIG. 20 below.

FIG. 20 illustrates more details of the embodiment in FIG. 19. In this Figure, cable 1600 is shown attaching to electrical circuitry 1900 though a center of the support bars 108, 110. Specifically, the cable 1600 has electrical wires that connect the cable 1600 to a plurality of electrical connections 2002, 2004, 2006, and 2008 within the electrical circuitry 1900. These electrical connections in turn connect to the extension 1800 that connects the support system 100 to the eye examination system (not shown).

Although this invention has been described with reference to an illustrative embodiment, this description is not intended to limit the scope of the invention. Various modifications and combinations of the illustrative embodiments as well as other embodiments of the invention will be apparent to persons skilled in the art upon reference to the description. It is therefore intended that the appended claims accomplish any such modifications or embodiments.

We claim:

1. An eye examination support device, comprising:
at least two support bars with attachments at a bottom of each of the at least two support bars;
a head rest attached to the at least two support bars;
a chin rest bar attached to the at least two support bars;
a chin rest attached on the chin rest bar;
at least two attachment areas at a bottom of the each of the at least two support bars, wherein the at least two attachment areas attach each of the support bars to a support table;
an electrical circuit that connects to the support device; and
an examination device positioned near the chin rest and the chin bar in order to allow an eye professional and/or technician to examine at least one eye of a patient, wherein the examination device electrically connects to the support device.

2. The support device of claim 1, wherein the chin rest adjusts vertically.

3. The support device of claim 1, wherein the chin rest is attached to the chin bar by an adjustment device to adjusts the chin rest vertically.

4. The support device of claim 1, wherein the chin bar is attached to one of the at least two support bars with an adjustment mechanisms that adjusts the chin bar vertically.

5. The support device of claim 1, wherein the adjustment mechanism includes a rotating handle that adjusts the chin bar vertically as the rotating handle is adjusted.

6. The support device of claim 1, wherein the examination device is attached to the support table.

7. The support device of claim 1, wherein each of the support bars includes a handle near the bottom of each of the support bars.

8. The support device of claim 7, wherein the handles on each of the support bars include a winged area that attaches the support table.

9. The support device of claim 7, wherein the handles attach to a support level and the support table, and wherein the examination device is attached to the support level.

10. An eye examination support device, comprising:
two support bar with attachments at a bottom of each of the two support bars;
a head rest attached to the two support bars;
a chin rest bar attached to the two support bars;
a chin rest attached on the chin rest bar;
two attachment areas at a bottom of the each of the two support bars, wherein the at least two attachment areas attach each of the support bars to a support table;
an electrical circuit that connects to the support device; and
an examination device positioned near the chin rest and the chin bar in order to allow an eye professional and/or technician to examine at least one eye of a patient, wherein the examination device electrically connects to the support device.

11. The support device of claim 10, wherein the chin rest adjusts vertically.

12. The support device of claim 10, wherein the chin rest is attached to the chin bar by an adjustment device to adjusts the chin rest vertically.

13. The support device of claim 10, wherein the chin bar is attached to one of the at least two support bars with an adjustment mechanisms that adjusts the chin bar vertically.

14. The support device of claim 10, wherein the adjustment mechanism includes a rotating handle that adjusts the chin bar vertically as the rotating handle is adjusted.

15. The support device of claim 11, wherein the examination device is attached to the support table.

16. The support device of claim 11, wherein each of the support bars includes a handle near the bottom of each of the support bars.

17. The support device of claim 16, wherein the handles on each of the support bars include a winged area that attaches the support table.

18. The support device of claim 16, wherein the handles attach to a support level and the support table, and wherein the examination device is attached to the support level.

19. An eye examination support device, comprising:
two support bar with attachments at a bottom of each of the two support bars;
a head rest attached to the two support bars;
an adjustable chin rest on a chin bar attached to the two support bars;
two handles near a bottom of the each of the two support bars, wherein the two handles include an attachment area that attach each of the support bars to a support table; an electrical circuit that connects to the support device; and
an examination device positioned near the chin rest and the chin bar in order to allow an eye professional and/or technician to examine at least one eye of a patient, wherein the examination device electrically connects to the support device.
